(19) United States
(12) Patent Application Publication
(10) Pub. No.: US 2021/0275429 A1
(43) Pub. Date: Sep. 9, 2021

(54) COSMETIC TREATMENT PROCESS FOR A KERATIN MATERIAL

(71) Applicant: L'OREAL, Paris (FR)

(72) Inventors: Jennyfer Cazares Delgadillo, Chevilly La Rue (FR); Dominique BORDEAUX, Chevilly La Rue (FR)

(21) Appl. No.: 16/482,888

(22) PCT Filed: Feb. 15, 2018
(86) PCT No.: PCT/EP2018/053809

§ 371 Date: Aug. 1, 2019

(30) Foreign Application Priority Data
Feb. 27, 2017 (FR) 1751565

(51) Int. Cl. A61K 8/81; A61K 8/73; A61Q 5/10; A45D 19/00

(52) U.S. Cl. CPC A61K 8/817; A61K 8/731; A61Q 5/10; A45D 19/0066; A61K 2800/5426; A45D 2200/25; A61K 2800/83; A61K 2800/87; A61K 2800/88; A45D 2200/20; A61K 2800/4324

(57) ABSTRACT

A cosmetic treatment process for a keratin material, including placing the keratin material in contact with at least one cationic polymer with a molecular weight of between 500 and 5,000,000 daltons, and either simultaneously with or consecutively to the placing the keratin material in contact with said cationic polymer, an electric current is applied using at least one electrode for a time that is sufficient to deposit at the surface of the keratin material an effective amount of said cationic polymer.

The present invention is directed toward proposing a treatment process, especially a cosmetic treatment process, for caring for and/or making up keratin materials, which is most particularly useful for improving the deposition or even the attachment of cationic polymers to the surface of a keratin material, and also to cosmetic treatment devices, especially makeup or care devices, for keratin materials, especially for performing the treatment process.

A wide diversity of compositions, especially cosmetic compositions, and more particularly compositions intended for caring for keratin materials, contain cationic polymers. These polymers are usually considered in these applications for the softness, smoothness and moisturization they afford to the target keratin material. With regard to their cationic nature, these polymers specifically have affinity for keratin materials that are naturally anionic. Consequently, they become attached thereto, usually via the establishment of bonds of van der Waals type, and then afford the desired softening, moisturizing and smoothing properties. The cationic polymers exploited in cosmetic compositions for these properties advantageously have a weight ranging from 500 to 5,000,000 daltons.

Thus, these cationic polymers are largely upgraded as conditioning agents with regard to keratin fibers. Compositions incorporating them are especially hair conditioners, which may be in the form of hair gels or lotions or more or less thick creams. Similarly, they are widely used in compositions intended for caring for and/or cleansing keratin materials such as the skin, where they are combined with detergent active agents and, precisely, compensate for the aggressive nature thereof. They may be, for example, shower gels or facial cleansing gels, this list not being limiting.

Unfortunately, these polymers attached to the surface of keratin materials are very often exposed to rinsing consecutive to their topical application. Now, this rinsing has the effect of partly removing these cationic polymers. Consequently, their beneficial effect is significantly reduced and above all not perpetuated over time. In addition, the cationic polymers removed by washing, which usually consists of rinsing with water, are conveyed with this water into the environment.

Finally, to overcome this partial removal of the cationic polymers, appreciable amounts of these compounds, generally ranging from 0.01% to 8% by weight of active material relative to the total weight of the composition, are generally considered in care compositions, which contributes toward exacerbating their environmental impact.

For all these reasons, it would be desirable to be able to optimize the efficacy of cationic polymers in smaller amounts. Together with this objective, it appears necessary to minimize the amounts of cationic polymers conveyed via the water into the environment.

The present invention is specifically directed toward meeting these objectives.

It has been found that the efficacy of cationic polymers can be significantly reinforced provided that a specific physical treatment of the target keratin material is considered together with or consecutive to their use.

Admittedly, lysis, iontophoresis or electrophoresis physical treatments have already been proposed in the cosmetic field.

Thus, the subject of WO 2006/008427 is a makeup process in which a tattoo is formed by electrophoresis. The pH, the conductivity and the density of the skin are not described.

As regards WO 94/17776, its subject is a process for applying a cationic Minoxidil derivative which is administered by iontophoresis to hair follicles, the cationic derivatives promoting hair growth.

However, neither of these two documents target an active agent within the meaning of the invention.

As regards WO 2009/131738, it describes a topical or cosmetic system which comprises a first element that is capable of acting as an electron donor and a second element that is capable of acting as an electron acceptor. Examples of electron-accepting components that are suitable for incorporation into the topical or cosmetic composition comprise cationic polymers. Said document, like the two preceding documents, therefore does target a final purpose in accordance with the present invention.

Treatment Process

Thus, the present invention relates mainly to a cosmetic treatment process for a keratin material, comprising the placing in contact of said keratin material with at least one cationic polymer with a molecular weight of between 500 and 5,000,000 daltons, wherein, simultaneously with or consecutive to the placing in contact of said cationic polymer with the keratin material, an electric current is applied using at least one electrode for a time that is sufficient to deposit at the surface of the keratin material an effective amount of said cationic polymer, in particular when electrically powered with a supply.

In one embodiment, the placing in contact of the cationic polymer with a keratin material and the application of the electric current are performed simultaneously.

In another embodiment, the step of placing a cationic polymer according to the invention in contact with a keratin material is performed first, followed by the step of applying the electric current.

The steps of placing a cationic polymer according to the invention in contact with a keratin material and of applying the electric current may be performed only once or several times.

The current applied, especially a unidirectional current, may be continuous or pulsed, or may comprise a continuous component to which is added a pulsed component.

As discussed previously, iontophoresis is a technique which consists in applying an electric current for the purpose of transporting charged molecules. More precisely, a low-intensity electric current is applied to a charged molecule preferably using an electrode of the same sign to produce a repellent effect which entrains the charged molecules away from the electrode toward a target. In certain embodiments, the effect of simple ionization (electromigration) is the main mechanism via which iontophoresis produces its transport properties. However, there are additional mechanisms known as electroosmosis and electroporation which are produced by an electric current. Electroosmosis induces a flow of solvent which transports uncharged molecules in the anode-cathode direction.

Iontophoresis is a technique of using an electric current that has already been selected for delivering molecules to a keratin material, whether the transportation of the molecule is by electromigration, electroosmosis or electroporation. However, this technique has only been considered for improving the intra-dermal penetration of a cosmetic or care active agent. In the context of the present invention, the objective pursued is entirely different. It has been found that the combination of an iontophoresis treatment, together with or consecutive to a topical application of cationic polymer(s) to a keratin material, makes it possible firstly to increase the degree of deposition and to promote the homogeneity of these polymers at the surface, and secondly to significantly increase the durability of this immobilization. In other words, the process according to the invention makes it possible to deposit more polymers homogeneously and to significantly reduce the amount of polymers removed in the course of consecutive rinsing.

This surprising result is advantageous in several respects.

Firstly, with regard to the gain in deposition of cationic polymers at the surface of keratin materials, by virtue of the invention it is possible to minimize the effective amount of cationic polymers in the usual formulations for treating keratin materials. Moreover, the environmental impact of these cationic polymers is also significantly attenuated by virtue of the reduction of the degree of removal of the deposited cationic polymers by the rinsing waters, thus making it possible to increase their efficacy over time and to reduce their use by excessively frequent treatments.

The process according to the invention also offers the advantage of allowing control of the degree of deposition of the cationic polymers via adjustment of the charge content (charge density) of these polymers and/or of the profile and/or intensity of the applied current.

Thus, in one implementation variant, the current intensity is modified especially for the purposes of locally adjusting the given degree of deposition of the cationic polymers on a keratin material.

The current intensity may be modified as a function of the location on the keratin materials. The current intensity may especially be varied according to the location on the hair from the root to the end.

For example, for application as a hair conditioner, the current is applied with a higher intensity at the ends of the hair, which are generally drier and need to be treated with an increased amount of cationic polymers than at the hair roots, thus overcoming the greasy and tacky appearance that may be generated by a high concentration of cationic polymers on contact with the scalp.

In one embodiment, the current intensity may be modified as a function of a locally detected characteristic, especially a color. It is possible, for example, to increase the current intensity locally, when a red spot is detected on the skin.

Finally, it is possible to exploit this increased degree of deposition of cationic polymers at the surface of keratin materials to give the keratin materials thus functionalized other properties via the interaction of these cationic polymers with the anionic form of one or more other additional active agents. According to this option, the treatment process according to the invention may comprise an additional step consecutive to the step of applying the electric current, the additional step comprising the placing in contact of the deposited cationic polymers with the anionic form of an additional cosmetic active agent and/or care active agent, chosen especially from dyestuffs or active agents for treating the skin.

In one embodiment, the steps of placing a cationic polymer according to the invention in contact with a keratin material and of applying the electric current are performed first, followed by the abovementioned additional step.

Hair coloring, skin coloring, a makeup result or a film deposit may thus be obtained.

The treated keratin materials may especially comprise the skin, the nails, the hair, the eyelashes, the eyebrows, bodily hair, the external mucous membranes of the human body, or the lips, this list not being limiting.

The invention does not concern penetration into these keratin materials. The object of the invention is to form a deposit on the keratin materials, for example in film form. By virtue of the invention, the regularity of the deposited film may be improved.

The process of the invention makes it possible especially to promote the moisturization, surfacing, protection or smoothing and softening of the skin, the moisturization, sheen, conditioning, disentangling, volume improvement or smoothing and softening of the hair, the fleshing of the lips, and the protection or softening of the areas of skin to be shaved or which have been shaved. The process according to the invention may also have a bactericidal effect.

The treated keratin materials are preferably negatively charged.

By way of example, the pH of the skin is above 5.0.

Preferably, the application of current in a process according to the invention is performed with a mean current density at the surface of contact with the keratin materials of between 0.01 mA/cm² and 0.5 mA/cm² rms (Root Mean Square), and preferably with a mean current density of 0.2 mA/cm² rms (Root Mean Square).

According to one embodiment, the process according to the invention comprises the application of a current, especially unidirectional, especially pulsed, preferably having a periodic waveform, for example sinusoidal, square, pulsed, sawtooth or triangular, or combinations thereof.

According to a particular embodiment, the current is applied for a time of between 30 seconds and 30 minutes, better still between 1 minute and 25 minutes, for example between 2 minutes and 22 minutes. In one embodiment, the current application time is, for example, 5 minutes. In another embodiment, this time is, for example, 20 minutes.

More particularly, in one example of implementation of the process in accordance with the invention, a continuous current and a pulsed current with a mean current density on the surface of the keratin materials ranging from 0.05 mA/cm² to 0.5 mA/cm² are simultaneously delivered; a pulse time ranging from 200 microseconds to 300 microseconds and a pulse frequency ranging from 100 Hz to 300 Hz.

According to a preferred embodiment, a process according to the invention comprises:

a) the generation of a current stimulus, especially a unidirectional continuous current with a mean current density of between 0.01 mA/cm² rms and 0.5 mA/cm² rms, and

b) the generation of a current stimulus, especially a unidirectional pulsed current with a mean current density of between 0.01 mA/cm² rms and 10 mA/cm² rms, a pulse time ranging from 10 microseconds to 500 microseconds; and a pulse frequency ranging from 10 Hz to 500 Hz;

the continuous current and the pulsed current being applied for a time that is sufficient for the surface deposition of said cationic polymer.

The process according to the invention may also comprise a step of pretreatment, which is thus prior to the steps of placing a cationic polymer according to the invention in contact with a keratin material and of applying the electric current, in which the polarity of the keratin materials is modified so as to increase the number of negative charges on the keratin materials, especially on the skin. Such a pretreatment step may make it possible to improve the subsequent deposition of said cationic polymer. This pretreatment step may make it possible to increase the pH of the skin, which may especially be advantageously brought above 5.0.

Device and Kit

A subject of the invention is also a cosmetic treatment device, especially for making up or caring for keratin materials, for performing the treatment process according to the invention, as defined above. The device may comprise means for applying an electric or electromagnetic current to the keratin materials.

The device may be used a first time by applying the composition to the keratin materials, with or without application of the electric current, and then one or more other times, without application of the composition but only with application of the electric current. Attachment of the cationic polymer to the keratin materials is thus promoted.

As a variant, the composition may be applied by hand, or using another applicator without the use of the device, followed by using the device to apply the electric current.

The device may comprise a composition reservoir, the composition comprising said at least one cationic polymer. The reservoir may especially contain at least an effective amount of at least one cationic polymer with a molecular weight ranging from 500 to 5,000,000 daltons.

The presence of the reservoir may make it possible to dispense the composition gradually as the application proceeds. The reservoir, which may be a removable cartridge, may comprise an inner space intended to receive the composition, or as a variant a substrate which may be porous and intended to be impregnated with the composition. The substrate may comprise a wick, a sponge, a sinter, a woven, a nonwoven or a knitted fabric, this list not being limiting.

The device may comprise an application electrode. The electrode is configured to allow the application and spreading of the cationic polymer on the keratin materials.

The device may also comprise a counterelectrode, which may be attached to a handle member of the device, or may be detached therefrom, and may be intended to be held in the user's other hand.

The device may also comprise a circuit operationally coupled to the application electrode and to the counterelectrode, which is configured to simultaneously generate at least one current stimulus, especially a unidirectional continuous and/or pulsed current to the application electrode, the current stimulus having an amplitude and a time sufficient to attach the cationic polymer.

The device may be configured for hair treatment. The device may especially have the form of a comb, which may comprise teeth having electrically conductive surfaces that can pass between the hairs. The gap between the teeth may especially be between 70 and 120 μm. The gap between the teeth may especially correspond to about the diameter of a human hair. Such a device may especially make it possible to stimulate the hair follicles with electric pulses. These pulses target the follicles, but prevent damage of the tissues surrounding them, thus avoiding any danger and any pain. The device may be used to promote the deposition of a cationic polymer onto the hair.

The device may be configured for treating the skin, and especially acne spots. The device may especially comprise a switch for the locally selective application of the electric current. This may make it possible more particularly to treat acne spots, and to avoid treating the rest of the skin.

Another subject of the present invention relates to a kit comprising:

a) a topical composition for caring for and/or washing keratin materials, comprising at least an effective amount of at least one cationic polymer, especially with a molecular weight ranging from 500 to 5,000,000 daltons, and

b) a device for treatment by application of an electric current, which is suitable for the implementation of a process in accordance with the invention, as defined above.

Cationic Polymers

For the purposes of the present invention, the term “cationic polymer” denotes any polymer containing cationic groups and/or groups that can be ionized into cationic groups.

The cationic polymers under consideration according to the invention have a molecular weight ranging from 500 to 5,000,000 daltons, preferably from 1000 to 3,000,000 daltons.

The cationic polymer may have a cationic charge density at least equal to 0.7, ranging from 0.9 to 7 meq/g and preferably from 0.9 to 4 meq/g.

The cationic charge density of a polymer corresponds to the number of moles of cationic charges per unit mass of polymer under conditions in which it is totally ionized. It may be determined by calculation if the structure of the polymer is known, i.e. the structure of the monomers constituting the polymer and their molar proportion or weight proportion. It may also be determined experimentally by the Kjeldahl method.

A composition that is suitable for use in the invention may comprise one or more cationic polymers of different chemical nature and/or of different charge density.

The composition may have a conductivity of between 0.1 and 50 mS/cm, better still between 0.5 and 25 mS/cm.

Thus, a composition may comprise one or more highly charged cationic polymers, i.e. polymers with a charge of greater than 4 meq/g and one or more weakly charged cationic polymers, i.e. polymers with a charge of less than 4 meq/g.

The cationic polymers under consideration according to the invention are chosen especially from:
homopolymers and copolymers obtained from one or more unsaturated monomers and comprising at least one quaternary ammonium group;
homopolymers and copolymers obtained from one or more unsaturated monomers and comprising a dimethyldiallylammonium radical;
quaternary copolymers of vinylpyrrolidone and of vinylimidazole;
quaternized polysaccharides;
chitosan or salts thereof, and mixtures thereof.

As illustrations of the homopolymers and copolymers obtained from one or more unsaturated monomers and comprising at least one quaternary ammonium group, mention may be made especially of:
(i) those described in French patents 2 505 348 and 2 542 997;

(ii) the cellulose ether derivatives containing quaternary ammonium groups, described in French patent 1 492 597, and in particular polymers sold under the names Ucare Polymer “JR” (JR 400 LT, JR 125 and JR 30M) or “LR” (LR 400 or LR 30M) by the company Amerchol [DC<4 meq/g].

These polymers are also defined in the CTFA dictionary as quaternary ammoniums of hydroxyethylcellulose that have reacted with an epoxide substituted with a trimethylammonium group.

(iii) cellulose copolymers or cellulose derivatives grafted with a water-soluble monomer of quaternary ammonium, and described especially in U.S. Pat. No. 4,131,576, such as hydroxyalkylcelluloses, for instance hydroxymethyl-, hydroxyethyl- or hydroxypropyl celluloses grafted, in particular, with a methacryloylethyltrimethylammonium, methacrylamidopropyltrimethylammonium or dimethyldiallylammonium salt.

The commercial products corresponding to this definition are more particularly the products sold under the names Celquat L 200 and Celquat H 100 by the company National Starch [DC<4 meq/g].

(iv) quaternary diammonium polymers constituted of repeating units corresponding to formula (I):

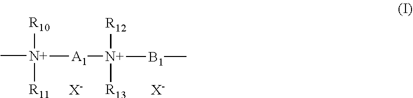

in which formula (I):

R₁₀, R₁₁, R₁₂ and R₁₃, which may be identical or different, represent aliphatic, alicyclic or arylaliphatic radicals containing from 1 to 6 carbon atoms or lower hydroxyalkylaliphatic radicals, or alternatively R₁₀, R₁₁, R₁₂ and R₁₃, together or separately, constitute, with the nitrogen atoms to which they are attached, heterocycles optionally containing a second heteroatom other than nitrogen, or alternatively R₁₀, R₁₁, R₁₂ and R₁₃ represent a linear or branched C₁-C₆ alkyl radical substituted with a nitrile, ester, acyl or amide group or a group —CO—O—R₁₄-D or —CO—NH—R₁₄-D where R₁₄ is an alkylene and D is a quaternary ammonium group;

A₁ and B₁ represent polymethylene groups containing from 2 to 8 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups, and

X⁻ denotes an anion derived from a mineral or organic acid;

A₁, R₁₀ and R₁₂ can form, with the two nitrogen atoms to which they are attached, a piperazine ring; in addition, if A₁ denotes a linear or branched, saturated or unsaturated alkylene or hydroxyalkylene radical, B₁ can also denote a —(CH₂)ₙ—CO-D-OC—(CH₂)ₙ— group, in which D denotes:
a) a glycol residue of formula: —O—Z—O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—
where x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula: —NH—Y—NH—, where Y denotes a linear or branched hydrocarbon-based radical, or else the divalent radical

—CH₂—CH₂—S—S—CH₂—CH₂—;
d) a ureylene group of formula: —NH—CO—NH—.

Preferably, X— is an anion such as chloride or bromide.

These polymers have a number-average molecular weight generally between 1000 and 100 000.

Polymers of this type are described especially in French patents FR 2 320 330, FR 2 270 846, FR 2 316 271, FR 2 336 434 and FR 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is more particularly possible to use polymers that are constituted of repeating units corresponding to formula (II) below:

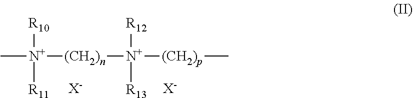

in which R₁₀, R₁₁, R₁₂ and R₁₃, which may be identical or different, denote an alkyl or hydroxyalkyl radical containing from 1 to 4 carbon atoms approximately, n and p are integers ranging from 2 to 8 approximately, and X— is an anion derived from a mineral or organic acid. Mention may be made in particular of Mexomer PO sold by the company Chimex [DC>4 meq/g].

(v) polyquaternary ammonium polymers formed from repeating units of formula (III):

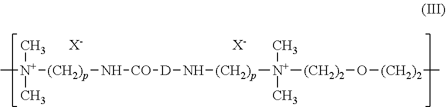

in which p denotes an integer ranging from 1 to 6 approximately, D may be zero or may represent a group —(CH₂)ᵣ—CO— in which r denotes a number equal to 4 or 7, and X⁻ is an anion.

Such polymers may be prepared according to the processes described in U.S. Pat. Nos. 4,157,388, 4,702,906 and 4,719,282. They are especially described in patent application EP-A-122 324.

Among these polymers, examples that may be mentioned include the products Mirapol A 15 [DC>4 meq/g], Mirapol AD1, Mirapol AZ1 and Mirapol 175 sold by the company Miranol.

As illustrations of the homopolymers and copolymers obtained from one or more unsaturated monomers and comprising a dimethyldiallylammonium radical, mention may be made most particularly of:

cyclopolymers of alkyldiallylamine or of dialkyldiallylammonium, such as the homopolymers or copolymers comprising, as main constituent of the chain, units corresponding to formula (IV) or (V):

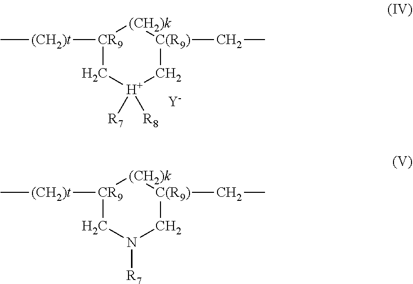

in which formulae k and t are equal to 0 or 1, the sum k+t being equal to 1; R₉ denotes a hydrogen atom or a methyl radical; R₇ and R₈, independently of each other, denote an alkyl group containing from 1 to 6 carbon atoms, a hydroxyalkyl group in which the alkyl group preferably contains 1 to 5 carbon atoms, or a lower (C₁-C₄) amidoalkyl group, or R₇ and R₈ may denote, together with the nitrogen atom to which they are attached, heterocyclic groups, such as piperidyl or morpholinyl; R₇ and R₈, independently of each other, preferably denote an alkyl group containing from 1 to 4 carbon atoms; Y— is an anion such as bromide, chloride, acetate, borate, citrate, tartrate, bisulfate, bisulfite, sulfate or phosphate. These polymers are in particular described in French patent 2 080 759 and in its Certificate of Addition 2 190 406.

Among the polymers defined above, mention may be made more particularly of the dimethyldiallylammonium chloride homopolymer sold under the name Merquat 100 (charge density of greater than or equal to 4 meq/g [DC=6.2]) by the company Lubrizol (and its homologs of low weight-average molecular mass) and the copolymers of diallyldimethylammonium chloride and of acrylamide, sold under the names Merquat 550 (charge density of less than 4 meq/g [DC=3.1]) and Merquat 7SPR (charge density of less than 4 meq/g [DC=3.056]).

As illustrations of quaternary copolymers of vinylpyrrolidone and of vinylimidazole, mention may be made especially of the products sold under the names Luviquat FC 905 (DC>4 meq/g), FC 550 (DC<4 meq/g) and FC 370 (DC<4 meq/g) by the company BASF. These polymers may also comprise other monomers, for instance diallyldialkylammonium halides. Mention may be made in particular of the product sold under the name Luviquat Sensation by the company BASF.

For their part, the quaternized polysaccharides may be chosen especially from quaternized guar gum, quaternized locust bean gum, quaternized xanthan gums, quaternized dextrins and quaternized starches.

More particularly, a cationic polymer according to the invention is chosen from homopolymers and copolymers obtained from one or more unsaturated monomers and comprising at least one quaternary ammonium group, homopolymers and copolymers obtained from one or more unsaturated monomers and comprising a dimethyldiallylammonium radical, and mixtures thereof.

More particularly, a cationic polymer according to the invention is chosen from:
cellulose ether derivatives comprising quaternary ammonium groups, and in particular the polymers sold under the names Ucare Polymer “JR” (JR 400 LT, JR 125 and JR 30M) or “LR” (LR 400 or LR 30M) by the company Amerchol [DC<4 meq/g], more particularly Polyquaternium-10 sold under the name Ucare Polymer JR-400 and
cyclopolymers of alkyldiallylamine or of dialkyldiallylammonium, such as the homopolymers or copolymers comprising, as main constituent of the chain, units corresponding to formula (IV) or (V) defined above, and in particular the dimethyldiallylammonium chloride homopolymer sold under the name Merquat 100 (charge density of greater than or equal to 4 meq/g [DC=6.2]) by the company Lubrizol (and its homologs of low weight-average molecular mass).

Preferably, the cationic polymer(s) according to the invention are chosen from Polyquaternium-6 and Polyquaternium-10, and more preferentially Polyquaternium-6.

According to a preferred embodiment, a cationic polymer used according to the invention is chosen from cyclopolymers of alkyldiallylamine or of dialkyldiallylammonium, such as the homopolymers or copolymers comprising, as main constituent of the chain, units corresponding to formula (IV) or (V) defined above, and in particular the dimethyldiallylammonium chloride homopolymer sold under the name Merquat 100 (charge density of greater than or equal to 4 meq/g [DC=6.2]) by the company Lubrizol (and its homologs of low weight-average molecular mass).

The total content of cationic polymer(s) in a composition that is suitable for use in the invention may range from 0.01% to 8% by weight relative to the total weight of the composition, preferably from 0.1% to 3% by weight and more preferentially from 0.2% to 2% by weight relative to the total weight of the composition.

In particular, the cationic polymers according to the invention with a cationic charge density of greater than or equal to 4 meq/g may be present in a content ranging from 0.01% to 5% by weight, preferably in a content ranging from 0.05% to 3% by weight and better still from 0.1% to 1.5% by weight relative to the total weight of the composition.

The cationic polymers according to the invention with a cationic charge density of less than 4 meq/g have a content ranging from 0.01% to 8% by weight, preferably a content ranging from 0.05% to 3% by weight and better still from 0.1% to 1.5% by weight relative to the total weight of the composition.

Composition Suitable for Use in the Invention

According to a preferential embodiment, a cationic polymer used according to the invention is conveyed in a care and/or washing composition, especially for keratin materials.

A composition according to the invention is advantageously administered topically to the intended keratin material.

Such a composition may be in the form of an aqueous, aqueous/alcoholic or oily solution, of a solution or dispersion of the lotion or serum type, of an emulsion with a liquid or semi-liquid consistency of the milk type, obtained by dispersion of a fatty phase in an aqueous phase (O/W) or vice versa (W/O), or of a suspension or emulsion with a soft, semi-solid or solid consistency of the cream type, of an aqueous or anhydrous gel, of a microemulsion, of a microcapsule, of a microparticle or of a vesicular dispersion of ionic and/or nonionic type.

These compositions are prepared according to the usual methods.

These compositions may especially constitute cleansing, protective, treatment or care creams, lotions, gels or mousses for caring for and cleansing the skin, mucous membranes, the scalp and/or keratin materials such as the hair.

They may be used for the cosmetic and/or dermatological treatment of the skin, the mucous membranes, the scalp and/or the keratin materials such as the hair, in the form of solutions, creams, gels, emulsions, mousses or else in the form of compositions adapted for use in an aerosol, for example also containing a pressurized propellant.

A composition according to the invention may advantageously be formulated in any galenical form that is suitable for haircare, especially in the form of a hair lotion in spray form, a shampoo, a conditioner, a detangler, a hair cream or gel, a styling lacquer, a hairsetting lotion, a treating lotion, a dye composition (especially for oxidation dyeing) optionally in the form of a coloring shampoo, a hair-restructuring lotion, a permanent-waving composition, a lotion or gel for combating hair loss, an antiparasitic shampoo or a medicated shampoo, especially an anti-seborrhea shampoo, a scalp care product, which is especially anti-irritant, antiaging or restructuring, or which activates the blood circulation.

In a known manner, the galenical forms intended for topical administration may also contain adjuvants that are common in the cosmetic and/or dermatological field, such as thickeners, oils, waxes, preserving agents, antioxidants, solvents, fragrances, fillers, UV screening agents and dyestuffs.

The amounts of these various adjuvants are those conventionally used in the field under consideration. Depending on their nature, these adjuvants may be introduced into the fatty phase and/or into the aqueous phase.

The composition of the invention may also advantageously contain water. The water may be a spring and/or mineral water, chosen especially from Vittel water, waters from the Vichy basin and la Roche Posay water. It may also be a deionized water.

The water may be present in a content ranging from 5% to 99% by weight, preferably ranging from 10% to 95% by weight and preferentially ranging from 15% to 95% by weight, relative to the total weight of the composition.

### DETAILED DESCRIPTION

The invention may be understood better from reading the following detailed description of nonlimiting exemplary embodiments thereof and from studying the appended drawing, in which:

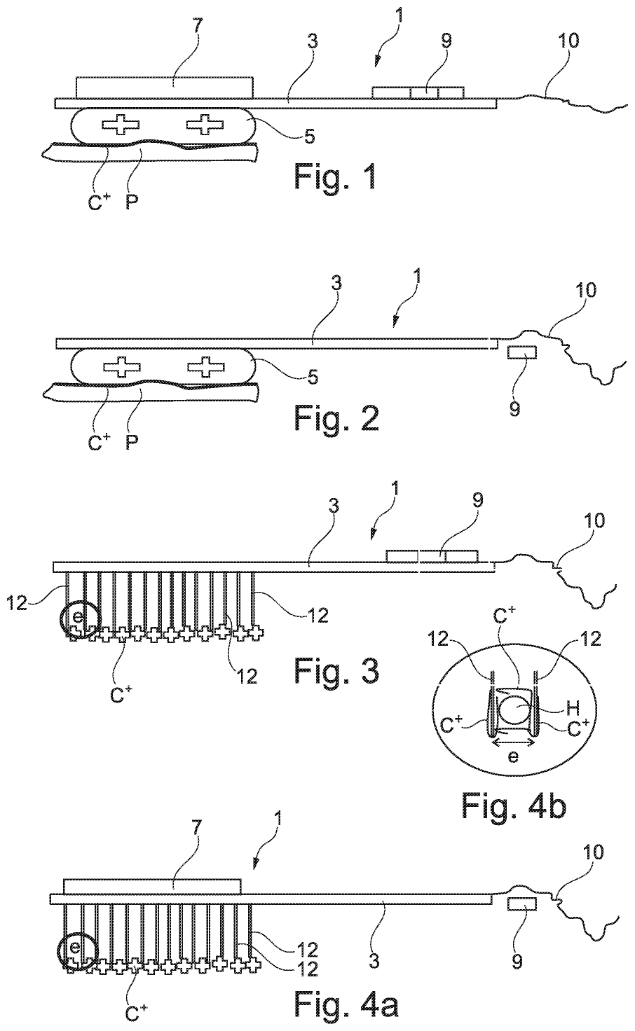

FIG. 1 is a schematic view of an example of a device according to the invention,

FIGS. 2 to 4 illustrate implementation variants,

FIG. 4a illustrates the deposition of a cationic polymer onto the hair, and

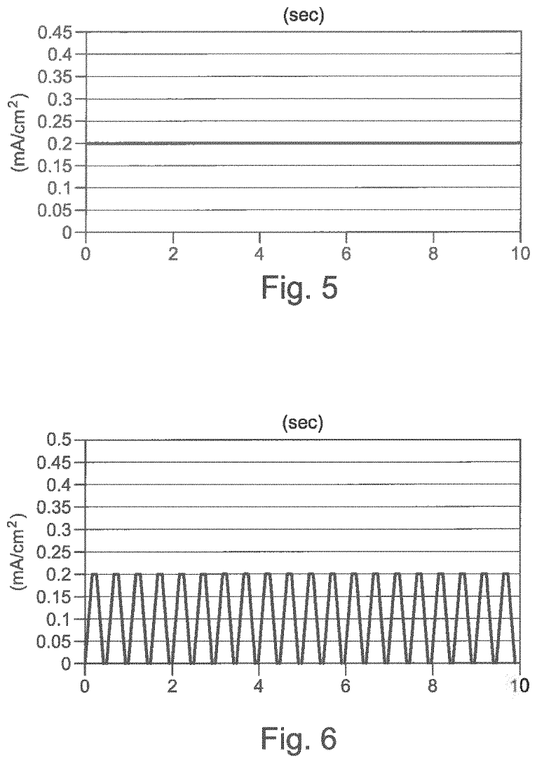

FIGS. 5 to 7 illustrate electric current stimuli according to various embodiments.

FIG. 1 shows a treatment device 1 for performing the process according to the invention on the skin P.

The device 1 comprises, in the described example, a handle member 3 bearing a positively charged application electrode 5 intended to allow the application of an electric current to the keratin materials, when the device is electrically powered by a supply 10, and also the application and spreading of the cationic polymer C+ onto the surface of the keratin materials P to be treated.

In the described example, the device 1 comprises a composition reservoir 7 allowing the application electrode 5 to be supplied with the composition. This reservoir may be in the form of a removable cartridge. The use of several cartridges of different compositions is possible. Thus, the composition may be dispensed gradually as it is applied to the keratin materials, especially the skin P.

The device 1 also comprises a counterelectrode 9, which is attached to the handle member 3 of the device 1.

As a variant, the counterelectrode 9 is intended to be held in the user's other hand, then being separate from the handle member 3 of the device 1 as illustrated in FIG. 2.

The embodiment illustrated in FIG. 2 also differs from that of FIG. 1 in that it lacks a reservoir 7, but comprises a porous, electrically conductive absorbent substrate intended to be impregnated with the composition, which goes with the application electrode 5.

Implementation examples for performing the process according to the invention on the hair H are illustrated in FIGS. 3 and 4. These devices are in the form of a comb comprising electrically conductive teeth 12 allowing the hairs to pass through so that the cationic polymer C+ is applied thereon. The gap e between the teeth may especially be between 70 and 120 μm, as illustrated in FIG. 4a, which shows a hair H in cross section.

In the embodiment of FIG. 3, the device comprises a counterelectrode 9 attached to the handle member 3 of the device 1. In addition, the device therein lacks a reservoir.

In the embodiment of FIG. 4, the device comprises a reservoir 7 for dispensing the composition on the teeth 12 and for allowing its application onto the hair. The counterelectrode 9 is separate from the handle member 3 of the device 1.

FIGS. 5-7 show embodiments of representative current density waveforms emitted by the iontophoresis device.

FIG. 5 shows a first current density waveform for the deposition of cationic polymers onto keratin materials via the use of iontophoresis.

The current density waveform is regulated about a constant value for the duration or a part of the iontophoresis treatment. A current density waveform regulated at a constant value is referred to as a continuous current, and the terms “constant current”, “galvanic current” and “continuous current” are interchangeable. The current density is defined by units in amperes per unit area (of the cross section of the active electrode).

Whereas FIG. 5 shows a certain value and a certain time for the continuous current density, it should be understood that the values shown are given for illustrative purposes. In one embodiment of the continuous current waveform of FIG. 5, the current density is constant and regulated at or below 0.5 mA/cm². In one embodiment of the continuous current waveform of FIG. 5, the current density is constant and regulated at or below 0.2 mA/cm². In one embodiment of the continuous current waveform of FIG. 5, the current density is constant and regulated between 0.01 mA/cm² and 0.5 mA/cm². In one embodiment of the continuous current waveform of FIG. 5, the current density is constant and regulated at any one of the following values: 0.01, 0.05, 0.1, 0.15, 0.2, 0.25, 0.3, 0.35, 0.4, 0.45, 0.5 mA/cm² or in a range between any two values serving as limit points. In one embodiment of the continuous current waveform of FIG. 5, the amplitude is constant at any one of the above values and the electric current is applied for a time of at least 1 minute. In one embodiment of the continuous current waveform of FIG. 5, the amplitude is constant at any one of the above values and the electric current is applied for a time of 10 to 20 minutes. In one embodiment of the continuous current waveform of FIG. 5, the amplitude is constant at any one of the above values and the electric current is applied for a time (in minutes) of 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60 or in any range between any two values serving as limit points.

FIG. 6 shows a second current density waveform for the deposition of cationic polymers onto keratin materials via the use of iontophoresis. The current density waveform is regulated as positive pulses. The pulses of FIG. 6 are single-phase, which means that the current is unidirectional. A pulse of FIG. 6 has a maximum amplitude. The pulse waveform will increase from a minimum amplitude, will reach the maximum amplitude and will then decrease to the minimum amplitude, will remain at the minimum amplitude, and the cycle will be repeated. A pulse is counted from the minimum amplitude until it reaches the maximum amplitude and then returns to the minimum amplitude. Thus, a pulse does not comprise the period of the minimum amplitude. A pulse cycle comprises the period at the minimum amplitude. When the pulse cycle frequency is indicated, the durations of the maximum and minimum amplitudes are not modified.

In one embodiment, the pulse wave is expressed so as to present a duty cycle percentage. In one embodiment, the expression of a duty cycle percentage relative to a pulse wave means that the electric current is on for the duty cycle percentage. For example, a 50% duty cycle means that the electric current is on for 50% and off for 50% of the pulse cycle, a 30% duty cycle means that the electric current is on for 30% and off for 70% of the pulse cycle. In one embodiment, the duty cycle percentage of unidirectional pulses is 5, 10, 15, 20, 25, 30, 35, 40, 45, 50, 55, 60, 65, 70, 75, 80, 85, 90, 95 or any range between two values serving as limit points. In one embodiment, the duty cycle percentage of biphasic pulses is 5, 10, 15, 20, 25, 30, 35, 40, 45, 50, 55, 60, 65, 70, 75, 80, 85, 90, 95 or any range between two values serving as limit points. In one embodiment, the pulse, meaning the “on” period, may be expressed as a duration having time units. In one embodiment, the “off” period of the pulse may be expressed as a duration. In one embodiment, the frequency of a pulse will be expressed in hertz, meaning cycles per second. In one embodiment, the pulses may be inverted by alternating the polarities of the first and the second electrode between positive and negative. In one embodiment, the biphasic pulses, the alternating pulses, the bidirectional pulses and the inverted pulses have the same meaning. In one embodiment, negative current density pulses will be followed by positive current density pulses, without remaining at a minimum. A pulse waveform comprising current density pulses that are both positive and negative will comprise a maximum value for the negative pulses, a maximum value for the positive pulses and the values must not be identical. In addition, in one embodiment, the duration of the negative current density pulse must not be the same duration as a positive current density pulse. In one embodiment, the duration of the pulses must not have the same duration, independently of whether the pulses are negative or positive.

In one embodiment, a pulse waveform may combine two or more pulse waveforms. In one embodiment, a pulse waveform may comprise negative pulses followed by positive pulses, thus having a maximum amplitude and a minimum amplitude for the negative pulses and a maximum amplitude and a minimum amplitude for the positive pulses.

Whereas FIG. 6 shows certain current density values of the maximum and minimum pulse amplitudes and pulse time values, it should be understood that the values shown are given purely for illustrative purposes. In one embodiment of the current waveform of FIG. 6, the current density is regulated as pulses and each pulse maximum is regulated at not more than 0.2 mA/cm² and the minimum amplitude is 0. In one embodiment of the current waveform of FIG. 6, the current density is regulated as pulses and each pulse maximum is regulated at or below 1 mA/cm² and the minimum amplitude is 0. In one embodiment of the current waveform of FIG. 6, the current density is regulated as pulses and each pulse maximum is regulated between 0.2 mA/cm² and 1 mA/cm² and the minimum amplitude is 0. In one embodiment of the current waveform of FIG. 6, the current density is regulated as pulses and each pulse maximum is regulated at or below 0.2 mA/cm² and the minimum amplitude is 0. In one embodiment of the current waveform of FIG. 6, the current density is regulated as pulses and each pulse maximum is regulated at 0.01, 0.05, 0.1, 0.15, 0.2, 0.25, 0.3, 0.35, 0.4, 0.45, 0.5 . . . mA/cm² or in the range between any two values serving as limit points.

In one embodiment, the pulse has a positive constant slope (different from the vertical) up to the maximum amplitude, followed by a time at the constant maximum amplitude, followed by a negative constant slope (different from the vertical) down to 0, followed by a time at 0. In one embodiment, the minimum may be other than 0. In one embodiment, the slope may be other than constant, for example exponential. In one embodiment of the current waveform of FIG. 6, the pulses are not triangular. In one embodiment of FIG. 6, the pulses are a square wave, the maximum and minimum amplitudes having the same duration. In one embodiment of the current waveform of FIG. 6, the pulses are a non-square wave. Certain embodiments of non-square waves comprise sinusoidal, sawtooth and triangular pulse waves, rectangular pulses, with exponential decrease and the like or corresponding combinations.

In one embodiment of FIG. 6, the duration of the maximum amplitude of the pulses is less than the duration of the minimum amplitude between the pulses. In one embodiment of FIG. 6, the duration of the maximum amplitude of the pulses is greater than the duration of the minimum amplitude between the pulses. In one embodiment of the current waveform of FIG. 6, the minimum amplitude is 0 mA/cm² (milliamperes per square centimeter). In one embodiment of FIG. 6, the minimum amplitude is greater than 0 mA/cm² (which means that it is positive relative to FIG. 6). In one embodiment of the current waveform of FIG. 6, the maximum (and minimum) amplitude may increase from one pulse to another. In one embodiment of the current waveform of FIG. 6, the maximum (and minimum) amplitude may decrease from one pulse to another. In one embodiment of the current waveform of FIG. 6, the maximum (and minimum) amplitude may increase from one pulse to another and then decrease from one pulse to another and repeat. In one embodiment of the current waveform of FIG. 6, the current density is regulated as pulses at any of the above values and the pulse frequency is from 1 Hz to 2000 Hz. In one embodiment of the current waveform of FIG. 6, the current density is regulated as pulses at any of the above values and each pulse cycle has a duration of between 5 microseconds and 500 milliseconds. In one embodiment of the current waveform of FIG. 6, the current density is regulated as pulses, the pulse frequency is 2000 Hz and each pulse has a duration of 250 microseconds.

In one embodiment of the current waveform of FIG. 6, the iontophoresis treatment is applied for a time of from 1 to 5 minutes. In one embodiment of the current waveform of FIG. 6, the iontophoresis treatment is applied for a time (in minutes) of 1, 1.5, 2, 2.5, 3, 3.5, 4, 4.5, 5 or in any range between any two values serving as limit points. In one embodiment of the pulse waveform of FIG. 6, the electric current is applied as pulses for a time (in minutes) of 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60 or any range between any two values serving as limit points.

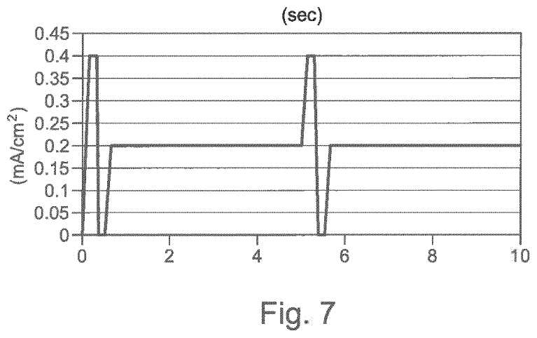

FIG. 7 shows a third current density waveform for the deposition of cationic polymers onto keratin materials via the use of iontophoresis. In one embodiment, the current density waveform is regulated as positive single-phase pulses or as biphasic pulses and intermediate pulses. In one embodiment, the current density is regulated as a constant continuous current. FIG. 7 shows a synchronous mode of application of two waveforms. The two waveforms in combination are applied synchronously for the duration or any part of the iontophoresis treatment. In particular, the pulse wave has an on period and an off period. By superposition of the pulse wave over the constant continuous current, the current profile shows the pulse which starts at the constant value of the constant continuous current. The pulse reaches a maximum for the predetermined time, then the profile returns to 0. After the off period, starting from the value 0, the constant continuous current is applied up to the next pulse. Thus, the current density of the waveform may be described as the addition of the constant continuous current of a first amplitude to a pulse of a second amplitude, the pulse having an off period before applying the continuous current again. A pulse is counted from the continuous current amplitude until it reaches the maximum pulse amplitude and then returns to the minimum amplitude or to 0. Thus, a pulse does not comprise the period of the minimum amplitude at 0. A pulse cycle comprises the period at the minimum amplitude. When the pulse cycle frequency is indicated, the durations of the maximum and minimum amplitudes are identical.

In one embodiment, the pulse is expressed so as to present a duty cycle percentage. In one embodiment, the expression of a duty cycle percentage relative to a pulse wave means that the electric current is on for the duty cycle percentage. For example, a 50% duty cycle means that the electric current is on for 50% and off for 50% of the pulse cycle, a 30% duty cycle means that the electric current is on for 30% and off for 70% of the pulse cycle. In one embodiment, the duty cycle percentage of unidirectional pulses is 5, 10, 15, 20, 25, 30, 35, 40, 45, 50, 55, 60, 65, 70, 75, 80, 85, 90, 95 or any range between two values serving as limit points. In one embodiment, the percentage of a respective biphasic pulse is 5, 10, 15, 20, 25, 30, 35, 40, 45, 50, 55, 60, 65, 70, 75, 80, 85, 90, 95 or any range between any two values as limit points. In one embodiment, the pulse, meaning the “on” period, may be expressed as a duration. In one embodiment, the “off” period of the pulse may be expressed as a duration. In one embodiment, the pulse wave will be expressed in hertz, meaning cycles per second. In one embodiment, the pulses may be inverted by alternating the polarities of the first and the second electrode between positive and negative. In one embodiment, the biphasic pulses, the alternating pulses and the inverted pulses have the same meaning. In one embodiment, negative current density pulses will be followed by positive current density pulses, without remaining at a minimum. A pulse waveform comprising current density pulses that are both positive and negative will comprise a maximum value for the negative pulses, a maximum value for the positive pulses and the values must not be identical. In addition, in one embodiment, the duration of the negative current density pulse must not be the same duration as a positive current density pulse. In one embodiment, the duration of the pulses must not have the same duration, independently of whether the pulses are negative or positive.

In one embodiment, a pulse waveform may combine two or more pulse waveforms. In one embodiment, a pulse waveform may comprise negative pulses followed by positive pulses, thus having a maximum amplitude and a minimum amplitude for the negative pulses and a maximum amplitude and a minimum amplitude for the positive pulses.

Whereas FIG. 7 shows certain current density values of the maximum and minimum pulse amplitudes and pulse time values, it should be understood that the values shown are given purely for illustrative purposes. In one embodiment of the current waveform of FIG. 7, the current density is regulated as pulses and each pulse maximum is regulated at not more than 0.2 mA/cm² and the minimum amplitude is 0. This means that the addition of the pulse to the continuous current is 0.4 mA/cm². In one embodiment of the current waveform of FIG. 7, the current density is regulated as a continuous current in combination with pulses and each pulse maximum is at or below 0.5 mA/cm² and the minimum amplitude is 0 and the continuous current is regulated at or below 0.5 mA/cm². In one embodiment of the current waveform of FIG. 7, the current density is regulated as a continuous current in combination with pulses and each pulse maximum is regulated between 0.2 mA/cm² and 0.5 mA/cm² and the minimum amplitude is 0 and the continuous current is regulated between 0.2 mA/cm² and 0.5 mA/cm². In one embodiment of the current waveform of FIG. 7, the current density is regulated as a continuous current in combination with pulses and each pulse maximum is regulated at or below 0.2 mA/cm² and the minimum amplitude is 0 and the continuous current is regulated at or below 0.2 mA/cm². In one embodiment of the current waveform of FIG. 7, the current density is regulated as a continuous current in combination with pulses and the continuous current and each pulse maximum is regulated at 0.01, 0.05, 0.1, 0.15, 0.2, 0.25, 0.3, 0.35, 0.4, 0.45, 0.5 mA/cm² or in the range between any two values serving as limit points.

In one embodiment of FIG. 7, the pulses are triangular with a defined maximum and a defined minimum, the maximum and minimum amplitudes having the same duration. In particular, the pulse has a positive constant slope (different from the vertical) up to the maximum amplitude, followed by a period at the constant maximum amplitude, followed by a negative constant slope (different from the vertical) down to 0, followed by a period at 0. In one embodiment, the minimum may be other than 0. In one embodiment, the slope may be other than constant, for example exponential. In one embodiment of the current waveform of FIG. 7, the pulses are not triangular. Certain embodiments of pulse waveforms comprise sinusoidal, sawtooth and square pulse waves, with exponential decrease and the like or corresponding combinations.

In one embodiment of FIG. 7, the duration of the maximum amplitude of the pulses is less than the duration of the minimum amplitude between the pulses. In one embodiment of FIG. 7, the duration of the maximum amplitude of the pulses is greater than the duration of the minimum amplitude between the pulses. In one embodiment of the current waveform of FIG. 7, the minimum amplitude is 0 mA/cm². In one embodiment of FIG. 7, the minimum amplitude is greater than 0 mA/cm² (which means that it is positive relative to FIG. 7) or negative relative to the continuous current (which means that its polarity is opposite to that of the continuous current). In one embodiment of the current waveform of FIG. 7, the maximum (and minimum) amplitude may increase from one pulse to another. In one embodiment of the current waveform of FIG. 7, the maximum (and minimum) amplitude may decrease from one pulse to another. In one embodiment of the current waveform of FIG. 7, the maximum (and minimum) amplitude may increase from one pulse to another and then decrease from one pulse to another and repeat.

In one embodiment of the current waveform of FIG. 7, the current density is regulated as a continuous current in combination with pulses at any of the above values and the pulse frequency is from 0.2 Hz to 500 Hz, with a corresponding pulse time of from 0.5 second to 0.0025 second. In one embodiment of the current waveform of FIG. 7, the pulse interval is 0.002 . . . 1.0, 1.5, 2.0, 2.5, 3.0, 3.5, 4.0, 4.5 or 5.0 seconds. In one embodiment of the current waveform of FIG. 7, the iontophoresis treatment is applied for a time of from 1 to 20 minutes.

In one embodiment of the current waveform of FIG. 7, the iontophoresis treatment is applied for a time (in minutes) of 1, 1.5, 2, 2.5, 3, 3.5, 4, 4.5, 5 or in any range between any two values serving as limit points. In one embodiment of the pulse waveform of FIG. 7, the electric current is applied as a continuous current and as pulses for a time (in minutes) of 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60 or any range between any two values serving as limit points.

In one embodiment of FIG. 7, the pulses are applied at a frequency of from 1 Hz to 200 Hz, each pulse being, respectively, from 1 second to 0.005 second. In one embodiment of FIG. 7, the pulses are applied at a frequency of 200 Hz, each pulse being 0.005 second.

In one embodiment, the current waveforms of FIGS. 5, 6 and 7 may be combined to give combinations of waveforms for the deposition of cationic polymers onto keratin materials via the use of iontophoresis. In one embodiment, the current waveform of any one of FIGS. 5-7 is applied for a first period, followed by a current waveform different from any one of FIGS. 5-7 for a second period, or vice versa. In one embodiment, two or more different current waveforms may be cycled throughout the treatment by an electric current. The particular features of the waveform as described above for FIGS. 2-4 are similarly applicable to combined treatments applying the two or more different waveforms. That is to say, any one or more of the embodiments of the continuous current waveform may be combined with any one or more of the pulse waveforms sequentially or simultaneously or with off period intervals.

In the case of the waveforms of FIGS. 5, 6 and 7, the maximum peak to peak voltage is 99 volts. In one embodiment the maximum electric current transmission time is 120 minutes during the iontophoresis treatment.

### EXAMPLES

Various tests were performed in order to evaluate in vitro the capacity of iontophoresis to increase the attachment of cationic polymers according to the invention to bodily hairs or the skin.

To do this, compositions comprising two different cationic polymers were applied according to the protocols detailed below to samples of hairy pig ear skin (the term “sample” is used hereinbelow to denote them) measuring 2 cm×2 cm, cleaned beforehand using a shampoo, or on skin samples.

The two cationic polymers tested are:
Polyquaternium-10 (Ucare® Polymer JR-400 sold by The Dow Chemical Company); and
Polyquaternium-6 (Merquat® 100 sold by Lubrizol).

### Example 1

A. Preparation and Treatment of the Bodily Hair Samples

a. Application of the Polymer

After cleaning the samples with shampoo, they are wetted with water, the excess water being removed manually.

The samples are then placed in a diffusion cell known as a Franz cell, and a magnetic stirrer is placed in the receptor compartment.

The edges of the donor and receptor compartments are then impregnated with a vacuum silicone (Rhodorsil silicones, Rhodia Siliconi) and the sample is placed between these compartments, the stratum corneum side of the sample facing the donor compartment.

Once the system has been attached using a clip, the receptor compartment is filled with 6 mL of an NaCl (150 mM)—HEPES (20 mM) pH 7.4 solution.

0.2 g of test polymer is then added to the donor compartment, followed by gentle massaging for 30 seconds by finger to impregnate the hairs with the test polymer.

A further 0.2 g of the polymer formulation tested is added to the donor compartment.

b. Applied Treatment

Each polymer formulation tested is left either for 5 minutes or for 20 minutes on the sample at 37° C. with magnetic stirring (200 rpm).

Furthermore, in each of these situations and for each of the formulations tested, either the formulation applied is allowed to diffuse passively, or the sample is subjected to anodic iontophoresis (0.5 mA/cm²-0.39 mA).

The sample subjected to iontophoresis is connected by means of a salt bridge to a flask containing 150 mM of NaCl and 20 mM of a HEPES buffer at pH 7.4.

The electrodes are then placed in the appropriate compartments: the anode in the flask connected to the donor compartment and the cathode in the receptor compartment.

The electrodes are connected to a KEPCO BHK-MG 0-2000V current-generating device from the company KEPCO, Inc., Flushing, NY(USA).

The current density and its intensity are indicated above.

The following eight treatments are tested:
composition with Polyquaternium-10 for 5 minutes without iontophoresis;
composition with Polyquaternium-10 for 5 minutes with iontophoresis;
composition with Polyquaternium-10 for 20 minutes without iontophoresis;
composition with Polyquaternium-10 for 20 minutes with iontophoresis;
composition with Polyquaternium-6 for 5 minutes without iontophoresis;
composition with Polyquaternium-6 for 5 minutes with iontophoresis;
composition with Polyquaternium-6 for 20 minutes without iontophoresis;
composition with Polyquaternium-6 for 20 minutes with iontophoresis.

After 5 or 20 minutes, the samples are rinsed with running water while passing the fingers between the hairs 10 times for 10 seconds. The excess water is removed manually or with a hairdryer (10 minutes at 60° C.).

c. Revelation of the Samples

A solution of Red 80 dye is prepared and used for the revelation of the samples.

The dye Red 80 is a water-soluble polyazo dye of direct type comprising 6 sulfonate functions. These anionic sites make it possible to reveal the cationic compounds present on the fiber. Thus, at the end of the experiment, the efficiency of attachment of the cationic polymers will be proportional to the intensity of the red color observed on the hairs of the treated sample.

A first solution prepared comprises:
0.4665 g of Red 80 dye;
0.125 mL of glacial acetic acid; and
a sufficient quantity (qs) of deionized water to make up to 100 mL.

The final solution applied to the samples comprises:
10.8 g of the Red 80 solution indicated above; and
qs of deionized water to make up to 54 g.

Each of the samples mentioned above is immersed in 1 mL of this final solution for 5 minutes without stirring, along with a sample which has undergone the same steps as the samples discussed above, except that it has not been placed in contact with a cationic polymer (negative control).

The samples are then rinsed five times with deionized water. Between each rinse, the samples are soaked in this deionized water for 1 minute.

Once these rinses have been performed, the excess water is removed manually and the samples are dried with a hairdryer at 70° C. for 15 minutes, before being observed.

B. Results

| Application time (min) | Without iontophoresis | With iontophoresis |
|---|---|---|
| 5 | 3 | 5 |
| 20 | 4 | 7 |

Results Obtained with Polyquaternium-10

| Application time (min) | Without iontophoresis | With iontophoresis |
|---|---|---|
| 5 | 7 | 8 |
| 20 | 7 | 10 |

Results Obtained with Polyquaternium-6

The values indicated in the above tables correspond to scores evaluated by observation with the naked eye, on a color intensity scale ranging from 1 (virtually no color observed) to 10 (highly colored).

No attachment of the dye Red 80 is observed on the control samples not exposed to a cationic polymer. Consequently, the color observed for the other samples may be attributed to the deposition of these polymers onto the hairs.

No substantial deposition is observed with Polyquaternium-10 in the absence of iontophoresis, either after 5 or 20 minutes of treatment. Conversely, a significantly stronger slight color is clearly observed after 5 minutes, and especially after 20 minutes, of treatment with application of iontophoresis.

With Polyquaternium-6, a slight color is observed after 5 or 20 minutes in the absence of iontophoresis. However, a significantly stronger color is observed in both cases when the hairs have been subjected to iontophoresis.

Consequently, irrespective of the cationic charge of the cationic polymer used, an improvement in the attachment of this polymer to the hairs is observed when they are subjected to the application of the electric current when compared with a treatment in the absence of a current. This improvement is also significant on the homogeneity of the observed deposit.

Moreover, the more positively charged the cationic polymer used, the greater the improvement in attachment imparted by the step of applying the current.

### Example 2

Skin samples are prepared and treated according to a methodology identical to that detailed in Example 1. These samples also consist, as indicated previously, of pig ear skin samples.

The results obtained are shown in the tables below:

| Application time (min) | Without iontophoresis | With iontophoresis |
|---|---|---|
| 5 | 5 | 5 |
| 20 | 5 | 5 |

Results Obtained with Polyquaternium-10

| Application time (min) | Without iontophoresis | With iontophoresis |
|---|---|---|
| 5 | 6 | 7 |
| 20 | 6 | 10 |

Results Obtained with Polyquaternium-6

The values indicated in the above tables correspond to scores evaluated by observation with the naked eye, on a color intensity scale ranging from 1 (virtually no color observed) to 10 (highly colored).

No attachment of the dye Red 80 is observed on the control samples not exposed to a cationic polymer. Consequently, the color observed for the other samples may be attributed to the deposition of these polymers onto the hairs.

No substantial deposition is observed with Polyquaternium-10 or Polyquaternium-6 in the absence of iontophoresis, either after 5 or 20 minutes of treatment. The skin is only slightly colored.

After treatment with application of iontophoresis, the behavior of Polyquaternium-10 on the skin is very different from that of Polyquaternium-6.

Specifically, no color difference is observed on the skin for Polyquaternium-10 after 5 minutes or 20 minutes of treatment with application of iontophoresis, relative to the color obtained in the absence of application of iontophoresis.

Conversely, a significantly stronger color is observed on the skin after 5 minutes or 20 minutes of treatment with application of iontophoresis for Polyquaternium-6, in particular after treatment of more than 5 minutes, in particular after a treatment of 20 minutes. This improvement is also significant on the homogeneity of the observed deposit.

These results confirm that the skin is much less negatively charged than the hairs. Results of post-treatment coloring with iontophoresis are thus indeed obtained, but with a polymer of the invention that is highly positively charged (Polyquaternium-6). A less positively charged polymer (Polyquaternium-10) thus has greater difficulty in being attached to a weakly negatively charged keratin material such as the skin during the application of the treatment with iontophoresis.

1. A cosmetic treatment process for a keratin material, comprising:
placing in the keratin material in contact with at least one cationic polymer with a molecular weight of between 500 and 5,000,000 daltons, and
either simultaneously with or consecutively to placing the keratin material in contact with said cationic polymer, applying an electric current using at least one electrode for a time that is sufficient to deposit at the surface of the keratin material an effective amount of said cationic polymer, when electrically powered with a supply.

2. The process as claimed in claim 1, wherein the application of the current is performed with a mean current density at the surface of contact with the keratin materials of between 0.01 mA/cm² rms and 1 mA/cm² rms.

3. The process as claimed in claim 1, wherein the current is applied for a time of between 30 seconds and 30 minutes.

4. The process as claimed in claim 1, comprising the simultaneous delivery of a continuous current and a pulsed current and the generation of a pulsed current stimulus with a mean current density on the surface of the keratin materials ranging from 0.05 mA/cm² rms to 0.5 mA/cm² rms, a pulse time ranging from 200 microseconds to 300 microseconds and a pulse frequency ranging from 100 Hz to 300 Hz.

5. The process as claimed in claim 1, wherein current intensity is modified for the purposes of locally adjusting a given degree of deposition of the cationic polymers on a keratin material.

6. The process as claimed in claim 1, wherein which the current intensity is modified as a function of location on the keratin materials, in which the keratin materials are hair and the current intensity is varied according to the location on the hair spanning from the root of the hair to the end of the hair.

7. The process as claimed in claim 1, wherein which the current intensity is modified as a function of a locally detected characteristic, especially a color.

8. The process as claimed in claim 1, further comprising an additional step consecutive to the step of applying the electric current, the additional step comprising placing in contact of the deposited cationic polymers with the anionic form of an additional cosmetic active agent and/or care active agent, chosen especially from dyestuffs.

9. The process as claimed in claim 1, wherein the cationic polymer has a cationic charge density at least equal to 0.7, ranging from 0.9 to 7 meq/g.

10. The process as claimed in claim 1, comprising:
a) the generation of a continuous current stimulus with a mean current density of between 0.01 mA/cm² rms and 0.5 mA/cm² rms; and
b) the generation of a current stimulus, especially a unidirectional pulsed current with a mean current density of between 0.01 mA/cm² rms and 10 mA/cm² rms, a pulse time ranging from 10 microseconds to 500 microseconds; and a pulse frequency ranging from 10 Hz to 500 Hz, the continuous current and the pulsed current being applied for a time that is sufficient to deposit said cationic polymer on the surface.

11. A device for the cosmetic treatment of keratin materials, for the use of the treatment process as claimed in claim 1, when the device is electrically powered by a supply.

12. A kit comprising:
a) a topical composition for caring for and/or washing keratin materials, comprising at least an effective amount of at least one cationic polymer with a molecular weight ranging from 500 to 5,000,000 daltons, and
b) a device for treatment by applying an electric current, which is suitable for performing a process as claimed in claim 1, when the device is electrically powered by a supply.
